(19) United States
(12) Patent Application Publication
(10) Pub. No.: US 2014/0314621 A1
(43) Pub. Date: Oct. 23, 2014

(54) METHODS AND DEVICES FOR TREATING SURFACES WITH SURFACE PLASMA

(71) Applicant: DREXEL UNIVERSITY, Philadelphia, PA (US)

(72) Inventors: GREGORY FRIDMAN, PHILADELPHIA, PA (US); ALEXANDER FRIDMAN, PHILADELPHIA, PA (US); ALEXANDER F. GUTSOL, SAN RAMON, CA (US); GENNADY FRIEDMAN, RICHBORO, PA (US)

(21) Appl. No.: 14/322,131

(22) Filed: Jul. 2, 2014

Related U.S. Application Data
(62) Division of application No. 13/256,727, filed on Dec. 21, 2011, now abandoned.
(60) Provisional application No. 61/160,564, filed on Mar. 16, 2009.

(51) Int. Cl. A61L 2/03

(52) U.S. Cl. CPC A61L 2/03
USPC 422/22

(57) ABSTRACT

Methods and devices for treating surfaces of objects using a non-thermal plasma are disclosed herein. The non-thermal plasma is generated through the use of an apparatus configured to generate a non-thermal plasma on its surface. The apparatus is comprised of a substrate that contains one or more electrodes of different polarity. The electrodes are placed within a layer of the substrate acting as a dielectric layer. The apparatus may also have additional layers to contain the dielectric layer. When an appropriate potential, being either an alternating current or pulsed high voltage potential, is applied and removed from the one or more electrodes, the gas on at least one surface of the apparatus becomes ionized and forms a non-thermal plasma. The electrodes can be configured to be of various shapes and sizes to modify or tune the plasma.

### CROSS REFERENCE TO RELATED APPLICATIONS

This application is a divisional of U.S. patent application Ser. No. 13/256,727, filed Dec. 21, 2011, which is a National Stage Entry of International Application No. PCT/US2010/27412, filed Mar. 16, 2010, which claims the benefit of U.S. Provisional Patent Application, Ser. No. 61/160,564, filed Mar. 16, 2009, the contents of which are hereby incorporated by reference in their entireties.

### GOVERNMENT RIGHTS

The subject matter disclosed herein was made with government support under Contract No. W81XWH-06-1-0742 awarded by the United States Army, USAMRMC. The Government has certain rights in the herein disclosed subject matter.

### TECHNICAL FIELD

The present subject matter is related the treatment of surfaces using plasma.

### BACKGROUND

Surfaces, such as human skin, medical devices, or appliances, may need to be treated for various reasons. For example, the area may need to be treated to sterilize the area against and/or from bacteria. If the area is to be sterilized, there are various ways in which to accomplish the sterilization. In a hospital or medical environment, a common way to sterilize equipment is to use an autoclave. An autoclave basically heats water in a pressurized environment, causing the production of steam at a temperature above steam produced at room pressure. The higher temperature steam acts on the instruments in the autoclave to sterilize the instruments. Other methods for sterilizing include the application of chemicals and radiation.

### SUMMARY

The present subject matter is directed to the treatment, including sterilization and/or disinfection, of surfaces using non-thermal plasma. The non-thermal plasma is generated on at least one surface of a substrate having two electrodes. In one aspect, the conductivity of the surface to be treated may vary, as the plasma is generated using two electrodes positioned within a flexible, semi-flexible or rigid substrate.

In one example, a device for treating surfaces is disclosed. The device is configured to generate a non-thermal non-equilibrium plasma on at least one surface of a substrate. A positive or high voltage electrode and a negative or ground electrode are at least partially disposed within the substrate such that, with the application of a potential having an alternating or pulsed current such that, at a certain potential and frequency, the two electrodes interact to cause the generation of the plasma on the surface of the substrate. Because the plasma is generated by the two electrodes within the substrate, the plasma is preferably formed independent of the type, morphology, shape, or material of a surface that may come in contact with the plasma.

In one exemplary and non-limiting example of the present subject matter, an apparatus for treating a surface is disclosed. In one example, the apparatus has a substrate with a first layer. The first layer acts as a dielectric. A first electrode is placed or disposed within the first layer and has, when a potential is applied to the first electrode, a first potential. The apparatus further has a second electrode that is placed or disposed in the first layer. The positioning of the first and second electrode within the first layer is configured to produce, when an appropriate voltage is applied to the first and/or second electrode, a non-thermal plasma generated on a surface of the apparatus. The apparatus may also have second and third layers that encapsulate, at least partially, the first layer. A surface of the second and third layers is laced against a surface of the first layer. Various electrical power may be supplied to the first or second electrode. In some configurations, the second electrode is grounded and the first electrode receives pulsed a high potential signal, positive or negative, from a power source.

In another exemplary and non-limiting example, a method of treating a surface is disclosed. An alternating current is applied to a first electrode or second electrode disposed in a first layer of a substrate. The first layer is configured to be a dielectric. The first layer has a first outer surface. When the alternating current is applied, the first electrode is at a first potential and the second electrode is at a second potential. When the power is removed, a non-thermal plasma is generated at the first outer surface. The plasma is applied to an object to be treated for a period of time. In some examples, the object is biological tissue which may or may not be alive or a medical device. Additional treatment may be provided by wrapping the apparatus around the object.

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

Other features of the subject matter are described below.

### BRIEF DESCRIPTION OF THE DRAWINGS

The summary, as well as the following detailed description, is further understood when read in conjunction with the appended drawings. For the purpose of illustrating the subject matter, there are shown in the drawings exemplary embodiments of the subject matter; however, the presently disclosed subject matter is not limited to the specific methods, compositions, and devices disclosed. In addition, the drawings are not necessarily drawn to scale. In the drawings:

FIG. 1 is a top-view illustration of an apparatus for treating a surface;

FIG. 2 is a side-view illustration of an apparatus for treating a surface;

FIG. 3 is an offset side-view illustration of an apparatus for treating a surface;

FIG. 4 is a side vide illustration of a semi-rigid or rigid apparatus for treating a surface;

FIG. 5a-5c are top-view illustrations of various interface shapes for the electrodes of an apparatus for treating a surface; and

FIG. 6 is an illustration of a flexible apparatus for treating a surface.

### DETAILED DESCRIPTION OF ILLUSTRATIVE EMBODIMENTS

The present subject matter may be understood more readily by reference to the following detailed description taken in connection with the accompanying figures and examples, which form a part of this disclosure. It is to be understood that this invention is not limited to the specific devices, methods, applications, conditions or parameters described and/or shown herein, and that the terminology used herein is for the purpose of describing particular embodiments by way of example only and is not intended to be limiting of the claimed invention.

Also, as used in the specification including the appended claims, the singular forms “a,” “an,” and “the” include the plural, and reference to a particular numerical value includes at least that particular value, unless the context clearly dictates otherwise. The term “plurality”, as used herein, means more than one. When a range of values is expressed, another embodiment includes from the one particular value and/or to the other particular value. Similarly, when values are expressed as approximations, by use of the antecedent “about,” it will be understood that the particular value forms another embodiment. All ranges are inclusive and combinable.

Plasmas, referred to as the “fourth state of matter,” are ionized gases having at least one electron that is not bound to an atom or molecule. In recent years, plasmas have become of significant interest to researchers in fields such as organic and polymer chemistry, fuel conversion, hydrogen production, environmental chemistry, biology, and medicine, among others. This is, in part, because plasmas offer several advantages over traditional chemical processes. For example, plasmas can generate much higher temperatures and energy densities than conventional chemical technologies; plasmas are able to produce very high concentrations of energetic and chemically active species; and plasma systems can operate far from thermodynamic equilibrium, providing extremely high concentrations of chemically active species while having a bulk temperature as low as room temperature.

Plasmas are generated by ionizing gases using any of a variety of ionization sources. Depending upon the ionization source and the extent of ionization, plasmas may be characterized as either thermal or non-thermal. Thermal and non-thermal plasmas can also be characterized by the temperature of their components. Thermal plasmas are in a state of thermal equilibrium, that is, the temperature of the free electrons, ions, and heavy neutral atoms are approximately the same. Non-thermal plasmas, or cold plasmas, are removed from a state of thermal equilibrium; the temperature of the free electrons is much greater than the temperature of the ions and heavy neutral atoms within the plasma.

The initial generation of free electrons may vary depending upon the ionization source. With respect to both thermal and non-thermal ionization sources, electrons may be generated at the surface of a cathode due to a potential applied across the electrode. In addition, thermal plasma ionization sources may also generate electrons at the surface of a cathode as a result of the high temperature of the cathode (thermionic emissions) or high electric fields near the surface of the cathode (field emissions).

The energy from these free electrons may be transferred to additional plasma components, providing energy for additional ionization, excitation, dissociation, etc. With respect to non-thermal plasmas, the ionization process typically occurs by direct ionization through electron impact. Direct ionization occurs when an electron of high energy interacts with a valence electron of a neutral atom or molecule. If the energy of the electron is greater than the ionization potential of the valence electron, the valence electron escapes the electron cloud of the atom or molecule and becomes a free electron according to:

e⁻+A→A⁺+e⁻+e^{-.}

As the charge of the ion increases, the energy required to remove an additional electron also increases. Thus, the energy required to remove an additional electron from A⁺ is greater than the energy required to remove the first electron from A to form A⁺. A benefit of non-thermal plasmas is that because complete ionization does not occur, the power to the ionization source can be adjusted to increase or decrease ionization. Similar and other effects may be achieved by, among other things, changing the rise time of the voltage signal or the waveform of the signal. This ability to adjust the ionization of the gas provides for a user to “tune” the plasma to their specific needs.

An exemplary thermal plasma ionization source is an arc discharge. Arc discharges have been otherwise used for applications such as metallurgy, metal welding and metal cutting and are known per se. Arc discharges are formed by the application of a potential to a cathode. Arc discharges are characterized by high current densities and low voltage drops. Factors relevant to these characteristics are the usually short distance between the electrodes (typically a few millimeters) and the mostly inert materials of the electrodes (typically, carbon, tungsten, zirconium, silver, etc). The majority of electrons generated in arc discharges are formed by intensive thermionic and field emissions at the surface of the cathode. That is, a much larger number of the electrons are generated directly from the cathode as opposed to secondary sources such as excited atoms or ions. Because of this intense generation of electrons at the cathode, current at the cathode is high, which leads to Joule heating and increased temperatures of the cathodes. Such high temperatures can result in evaporation and erosion of the cathode. The anode in arc discharges may be either an electrode having a composition identical or similar to the cathode or it may be another conductive material. For example, the anode in arc discharges used in metal welding or cutting is the actual metal be welded or cut.

Although thermal plasmas are capable of delivering extremely high powers, in addition to the electrode erosion problems discussed above, thermal plasmas have additional drawbacks. For example, thermal plasmas do not allow for adjusting the amount of ionization, and thus, they operate at extremely high temperatures. This limits the applications in which thermal plasma may be used to systems that either can withstand the temperatures associated with thermal plasmas or systems having replaceable structures that are damaged by the high temperatures.

Non-thermal plasma ionization sources have alleviated some of the above-mentioned problems. Exemplary ionization sources for non-thermal plasmas include glow discharges, dielectric barrier discharges, and gliding arc discharges, among others. In contrast to thermal plasmas, non-thermal plasmas provide for high selectivity, high energy efficiencies, and low operating temperatures. In many non-thermal plasma systems, electron temperatures are about 10,000 K while the bulk gas temperature may be as cool as room temperature.

In one example of a non-thermal plasma, dielectric barrier discharge (DBD) may be utilized using an alternating current at a frequency of from about 0.5 kHz to about 500 kHz between a high voltage electrode and a ground electrode. It should be noted that in certain configurations, a single pulse may be used. Therefore, the present subject matter may be preferably used in applications ranging from a single pulse to about 500 kHz. In addition, one or more dielectric barriers are placed between the electrodes. DBDs have been employed for over a century and have been used for the generation of ozone in the purification of water, polymer treatment (to promote wetability, printability, adhesion), and for pollution control. DBDs prevent spark formation by limiting current between the electrodes. Sample surface power density outputs may be between about 0.001 Watt/cm² to about 100 Watt/cm².

Various materials can be utilized for the dielectric barrier. These include plastic, glass, quartz, and ceramics, among others. The clearance between the discharge gaps is typically between about 0.01 mm and several centimeters. The required voltage applied to the high voltage electrode varies depending upon the pressure and the clearance between the discharge gaps. For a DBD at atmospheric pressure and a few millimeters between the gaps, the voltage required to generate a plasma may vary, but in some configurations, is about 10 kV.

In the present subject matter, the high voltage electrode and the ground electrode are at least partially encapsulated within a substrate which acts as the dielectric barrier, or dielectric. In a configuration in which the substrate is a solid, the electrical field generated by the two electrodes interacts not between a gap between the two electrodes but rather interacts with a gas in contact with a surface of the dielectric barrier. The gas ionizes in a manner conducive to the formation of a plasma and a power source is configured to create the electrical conditions for a non-thermal plasma. Because the plasma that is generated does not appreciably increase the temperature of the surface exposed to the plasma, it may be used for various purposes such as, but not limited to, disinfection and/or sterilization.

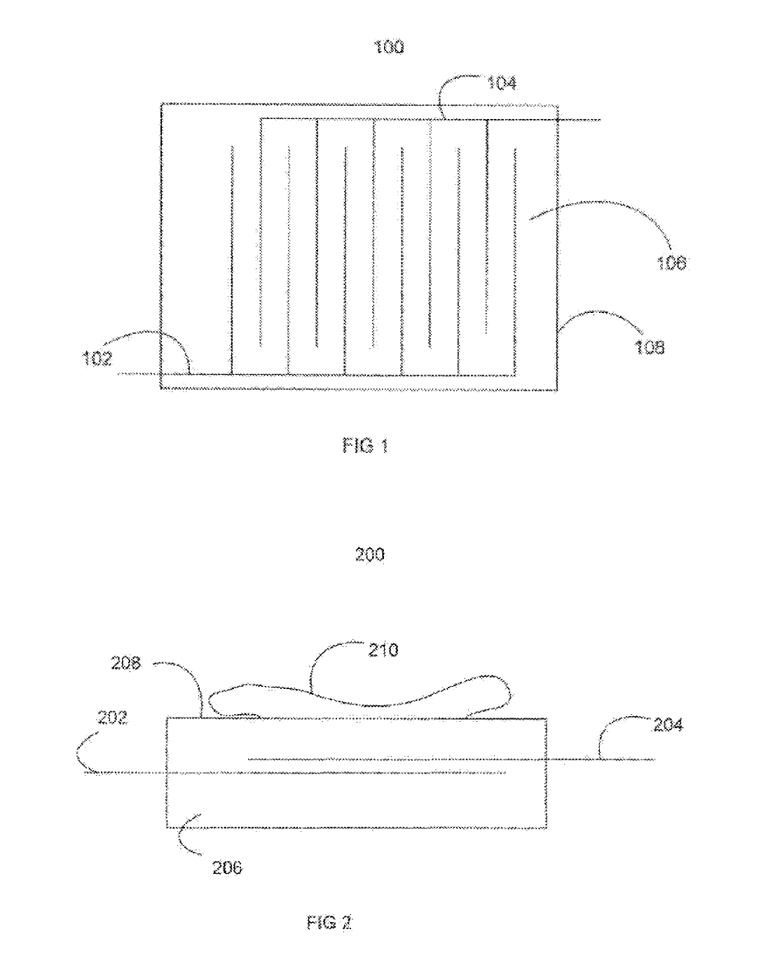

Referring now to the drawings, wherein like reference numerals designate corresponding structure throughout the views, and referring in particular to FIG. 1, an exemplary illustration of an apparatus, device 100, for treating surfaces is described. Device 100 has within a substrate, or more than one substrates, two electrodes of opposite potential in electrical communication with a power source. Thus, because device 100 is essentially a self-contained unit, with both a first and second electrode of opposite polarities, a second electrode in addition to device 100 is not necessary.

More specifically, shown in FIG. 1 is an overhead view of device 100. Device 100 has electrodes 102 and 104 at least partially contained within substrate 106. Electrodes 102 and 104 are in electrical communication with an alternating current or pulsed power supply (not shown) that provides a preferable electrical potential to electrodes 102 and 104. Electrodes 102 and 104 are positioned within substrate 106 at an appropriate position to generate a non-thermal plasma at the surface of device 100. Electrodes 102 and 104 are electrically connected to opposite polarities of the power supply (not shown).

The position of electrodes 102 and 104 within substrate 106 depends upon various factors, including the type of substrate used which affects the dielectric properties of substrate 106 and the electrical potential applied to electrodes 102 and 104 which affect the electrical properties of device 100. Additionally, if the position of electrodes 102 and 104 within substrate 106 of device 100 are set based upon other criteria or are otherwise not modifiable, the potential applied to device 100 may be changed to generate a surface plasma.

FIG. 2 is an exemplary side-view illustration of a device for treating surfaces. As illustrated, device 200 has at least partially contained within its structure electrode 202 and electrode 204, each connected to an electrical power source (not shown). As discussed earlier with regards to FIG. 1, electrode 202 and electrode 204 are connected to the opposite polarities of the power source. It should be understood that illustrating only one electrode for each polarity of the power source is only for purposes of illustration and not intended to limit the scope of the present subject matter to a single positive and a single negative electrode configuration. Further, as discussed previously, the power supplied, which is preferably a high potential signal, to device 200 may vary from a pulsed power source to an alternating current power source, depending upon the use and configuration of device 100.

Shown further is dielectric 206, or substrate. Dielectric 206 may be comprised of various substances, including ceramics and plastics, depending upon various factors, such as the intended use. For example, if the intended use is medical applications, in which a sterile, rigid, and non-porous substance may be needed, dielectric 206 may be comprised of various plastics or other materials suitable for use in a medical environment and may include, but are not limited to, low density polyethylene, high density polyethylene and polyvinyl chloride. In another example, in which device 200 may need to be flexible to accommodate a surface of varying shape, dielectric 206 may be comprised of flexible insulation such as the type that would be used to insulate wires or silicone-based substrates. Further, it should be understood that electrode 202, or electrode 204, or both, may be fully or partially encapsulated by dielectric 206.

If an alternating current potential is used, when alternating electrical power is applied to electrodes 202 and 204 (each being of different polarity), the gas in contact with or in close proximity to surface 208 ionizes in a manner that is conducive to the formation of a non-thermal plasma. The plasma that is generated, plasma 210, is preferably generated on surface 208 of device 200. In certain configurations of electrical power and frequency, plasma 210 is a non-thermal plasma in contact with or in close proximity to surface 208. Because of the energy within plasma 210, plasma 210 can be used for a variety of applications such at the treatment of surfaces to partially or fully sterilize the surface. For example, plasma 210 of device 200 may be used to treat, e.g. sterilize or disinfect, a wound, skin, or instrument such as a scalpel. Because the plasma is non-thermal, the temperature of the surface being treated does not appreciably change (preferably by only a couple of degrees), and thus, plasma 210 may be used on a variety of surfaces.

The size and shape of plasma 210 are related to the power supplied to device 200 and the location of electrodes 202 and 204 within dielectric 206. It should be noted that plasma 210 may be formed on various surfaces of device 200 and may in certain configurations fully envelop device 200. Further, it should be understood that, depending upon the configuration of device 200, including the size, shape and position of electrodes 202 and 204, dielectric 206 and the power supplied to device 200, that some of device 200 may also become ionized and may create within device 200 a plasma.

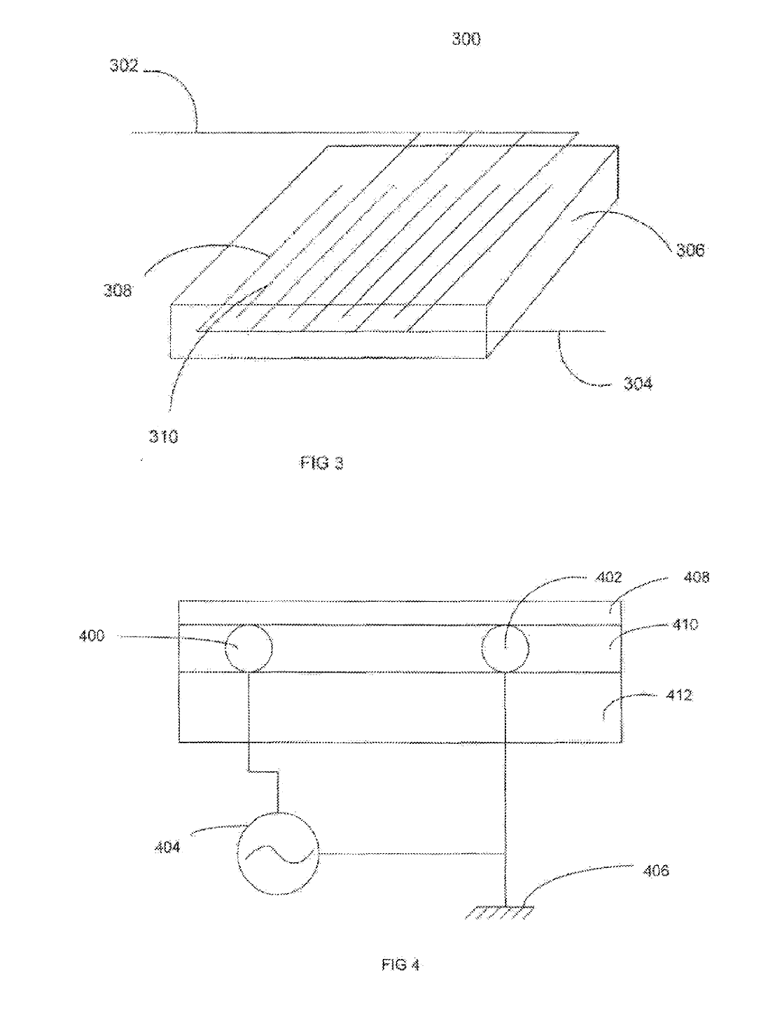

FIG. 3 is an elevated side view of device 300 configured to generate a surface plasma. Device 300 is comprised of first electrode 302 and second electrode 304, with second electrode 304 being of opposite polarity, or ground, to first electrode 302. Electrodes 302 and 304 are contained, at least partially, within substrate 306. In the configuration of device 300 shown in FIG. 3, electrodes 302 and 304 are comprised of multiple electrical connectors disposed in parallel. For example, electrical connector 308 is part of electrode 302 and is disposed within substrate 306 in essentially a parallel configuration to electrical connector 310. As discussed above, there may be more than one electrode 302 and/or electrode 304. Further, electrodes 302 and 304 may have various sizes and shapes that provide for, among other things, the ability to shape and size the plasma formed on one or more surfaces of device 300. For example, in the configuration shown in FIG. 3, electrodes 302 and 304 may be used to create a plasma on a greater surface area of device 300 than what may be attainable using only a single wire.

FIG. 4 is an exemplary electrical circuit that may be used to provide for the alternating current to generate a surface plasma. In FIG. 4, electrode 400 and 402 are disposed within dielectric 410. In the device of FIG. 4, dielectric 410 is also disposed within substrate 408 and substrate 412. The benefits of putting dielectric 410 between substrates 408 and 412 may include, but are not limited to, the ability to enhance or tune the plasma for a particular use. Because a plasma is generated by ionizing a gas, the manner in which the electrical field is generated affects the ionization of the gas, which in turn affects the plasma.

By changing the configuration of a device for generating a surface plasma, such as having dielectric 410 disposed between substrate 408 and 412, the plasma that is formed on a surface may be changed. For example, substrate 412 may be a conductive metal that does not allow the formation of electrical charges on its surfaces whereas substrate 408 may be glass that does. In that configuration, the device may be configured only to generate plasma on the surface of substrate 408 rather than the surface of substrate 412.

Electrodes 400 and 402 are in electrical communication with alternating current power source 404. Power source 404 generates an AC electrical signal that provides power to electrodes 400 and 402. In the configuration of FIG. 4, electrode 402 is grounded to ground 406 whereas electrode 400 receives the alternating positive then negative voltage. Upon application of an alternating voltage from power source 404 to electrode 400, gases surrounding various surfaces may be ionized and generate a plasma.

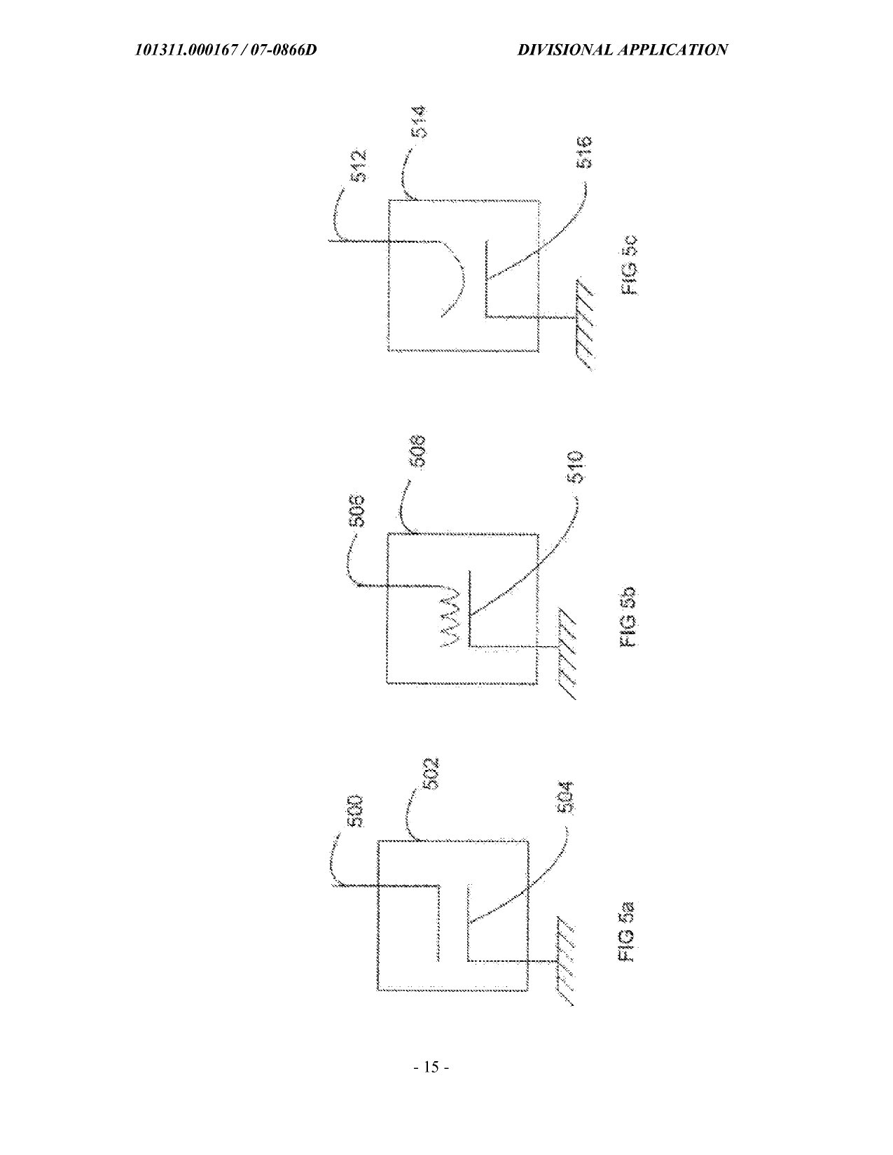

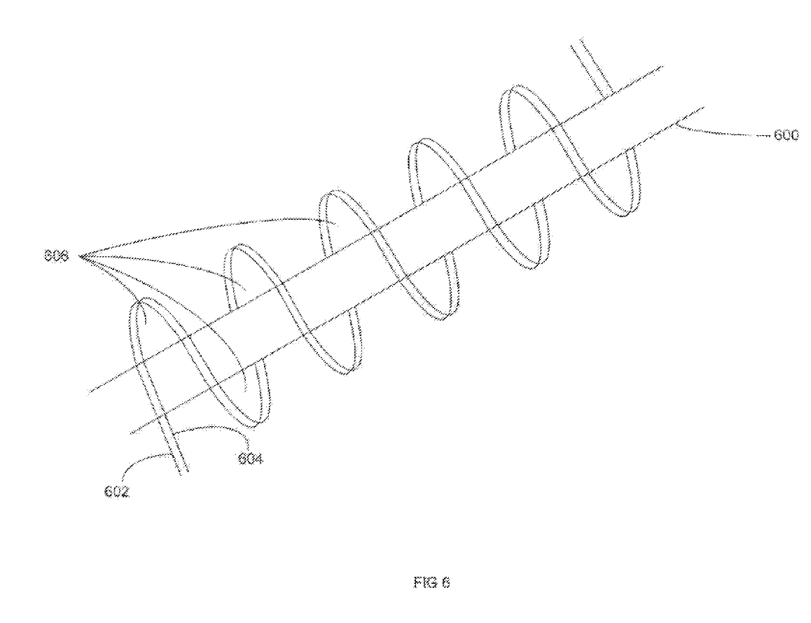

As discussed previously, the electrodes of the present subject matter, such as electrodes 400 and 402 of FIG. 4, may be shaped and sized in various configurations to modify or tune the plasma being generated. FIGS. 5a-5c illustrate exemplary shapes of electrodes that may be used. For example, FIG. 5a has electrode 500 and electrode 504 disposed within substrate 502. Electrodes 500 and 504 are shaped to have a parallel electrode configuration. In FIG. 5b, electrodes 504 and 510 are disposed within substrate 508 and are shaped to have a point, electrode 506 to plane, electrode 510, configuration. In FIG. 5c, electrodes 512 and 516 are disposed within substrate 514 and are shaped to have a circle, electrode 512, to plane, electrode 516, configuration. Other shapes and configurations may be used in conjunction with or in lieu of varying the electrical power source output to configure the plasma generated.

Further, the shape of a device for generating a surface plasma may also be configured for various applications. FIG. 6 illustrates a device for generating a surface plasma in which the shape of the device is modified to take on the shape of a surface being treated. In the illustration of FIG. 6, component 600 is a cylindrical rod having a surface that is to be treated with the surface plasma. For example, component 600 may be a catheter tube on the arm of a human patient for use in a surgical procedure. It may be desirable to constantly treat the surface of component 600 to maintain a sterile environment on the surface to prevent or reduce the possibility of infection brought into the body by viruses or bacteria present on the surface of component 600.

To fully treat the surface of component 600, it may be necessary to wrap around the surface a device for treating the surface with plasma. A two conductor wire having wires 602 and 604 are wrapped around component 600 (substrate or insulation not shown). An electrical power source is applied to wire 602 with wire 604, which act as electrodes, acting as a ground wire. Plasma is generated at various points of the device, shown for exemplary purposes only as areas 606. Thus, the surface of component 600 may be sterilized. It should be understood that other shapes and configurations may be used to treat various surface. For example, instead of having both electrodes within a substrate, such as the wire insulation (not shown) surrounding wire 602 and wire 604, wire 604 may not be used and the power source may be configured to generate the electric field on the surface of the substrate without the use of the second electrode. For example, a single conductor wire may be used.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment but rather should be construed in breadth and scope in accordance with the appended claims.

What is claimed:

1. A method of treating an object comprising:
applying high potential signal to a first electrode or a second electrode, wherein the first electrode and the second electrode are disposed within a first layer of a substrate configured to be a dielectric, wherein the substrate further comprises a first outer surface, the high potential signal giving rise to the first electrode having a first potential disposed in the first layer, and a second electrode having a second potential disposed in the first layer, the high potential signal giving rise to a non-thermal plasma to be generated at the first outer surface; and
applying the non-thermal plasma to the object for a period of time.

2. The method of claim 1, wherein the object is biological tissue or a medical device.

3. The method of claim 1, further comprising wrapping the apparatus around the object.

4. The method of claim 1, wherein the high potential signal is a pulsed power source or an alternating current power source.

5. The method of claim 4, wherein the frequency of the alternating current power source or pulsed power source is in a range of from about 0.5 kHz to about 500 kHz.

6. The method of claim 4, wherein the power output of the alternating current power source or pulsed power source is in a range of from about 0.5 Watt/cm² to about 2 watt/cm².

7. The method of claim 1, wherein first layer of a substrate comprises glass, quartz, ceramic, or plastic.

8. The method of claim 1, wherein the first layer of a substrate is flexible insulation.
